(19) United States
(12) Patent Application Publication
(10) Pub. No.: US 2008/0221572 A1
(43) Pub. Date: Sep. 11, 2008

(54) DEVICE FOR TEMPORARY FIXATION OF PARTS OF A HUMAN JOINT

(75) Inventors: Hans Naegerl, Gleichen (DE); Dietmar Kubein-Meesenburg, Goettingen (DE); Caspar Graf Stauffenberg, Gauting (DE)

Correspondence Address: DARBY & DARBY P.C., P.O. BOX 770, Church Street Station, New York, NY, 10008-0770, US

(73) Assignee: AEQUOS Endoprothetik GmbH, Graefelfing (DE)

(21) Appl. No.: 12/045,419

(22) Filed: Mar. 10, 2008

(30) Foreign Application Priority Data
Mar. 8, 2007 (DE) 10 2007 011 581.6

(51) Int. Cl. A61B 17/00

(52) U.S. Cl. 606/57; 606/54

(57) ABSTRACT

A device for temporary fixation of parts of a human joint includes two holding elements disposed opposite one another and a releasable fixing device configured to fix in place a respective joint part of the human joint between the two holding elements. In addition, the device includes a support surface configured to support a distal joint part and a proximal joint part, a guide configured to position and to limit a relative movement of at least one of a surgical instrument and a surgical aid, and a receiving means for receiving the guide.

Priority is claimed to German Patent Application No. DE 10 2007 011 581.6, filed Mar. 8, 2007, the entire disclosure of which is incorporated by reference herein.

The present invention relates to a device for temporary fixation of parts of a human joint in the hand and finger region, the device including two holding elements which are disposed opposite one another and between which the respective part of the joint can be fixed in place by means of a releasable fixing means.

### BACKGROUND

Such a device is also referred to as an external fixator and is attachable externally through the skin and used as a support system to temporarily fix a joint in a predetermined position, for example, in order to immobilize a bone fracture. More specifically, the fixation device is anchored on both sides of the bone. After the bone is set, it is held in the predetermined position by a rigid device, which may be in the form of rods. Thus, the device contributes to the healing process.

An external fixator is used, for example, for open fractures with soft tissue injuries, for comminuted fractures and, occasionally, in the case of pseudarthroses. It may also be used to deliberately stiffen joints (a procedure referred to as arthrodesis). An example of this would be the use of an external fixator across the knee joint.

A finger fixator of this type is described in DE 198 56 890 A1. The finger fixator disclosed therein has the degrees of freedom of movement that are required of a joint in a broken finger in order to prevent stiffening of the joints, especially in the case of comminuted fractures in or near the joint. This finger fixator is intended to allow a joint in a broken finger to be moved in a precise, definable and reproducible manner without any subjective influence. To this end, two rod-shaped articulated elements are used to which distal and proximal bone fixation elements may be secured in fixed relationship therewith. The distal and proximal bone fixation elements are positioned on a broken finger on both sides of the joint. The two rod-shaped elements are connected to at least one mechanical drive mechanism, allowing the distal rod-shaped element to pivot about a center or axis of rotation located at least near the center of rotation of the joint of the finger.

German Patent Document DE 197 54 529 A1 relates to a finger fixator which is used to distract individual fingers along a circular path and is connected by bone screws to a distal part and a proximal part of a finger. This invention is intended not only to allow lengthening of an injured finger, but to also enable shaping of the injured finger with only one surgical procedure; the uniform bending making it possible to significantly improve the basic function of the finger.

European Patent Document EP 06 85 206 A1 also describes an external fixator for treatment of fractures in the hand and finger region. The wires used herein may be conventional Kirschner wires which are each held directly between two nuts on a threaded rod. The transverse openings intended for this purpose are in the form of longitudinal slots having a thickness not significantly greater than that of the wires.

Other devices for treatment of fractures in the hand and finger region are disclosed in documents FR 2 855 040 A1, JP 11290338 A, DE 43 25 173 C1 and U.S. Pat. No. 6,063,087 A.

### SUMMARY OF THE INVENTION

It is an aspect of the present invention to improve a device for temporary fixation of parts of a human joint in the hand and finger region in such a manner that, in particular, surgical treatment becomes much easier.

The device of the present invention has a support surface for the distal joint part and the proximal joint part, and a receiving means for at least one guide against which a surgical instrument or a surgical aid can be positioned to limit relative movement. This provides a simple and safe way of treatment without a high degree of complexity. The device of the present invention allows the joint parts of the hand and finger region to be worked on in a reliable and precise manner in nearly any type of surgery, for example, also in surgical procedures where one or more bones is/are deliberately cut and fixed in a new position. Thus, the alignment of the surgical instrument is no longer exclusively dependent on the dexterity of the operator. Rather, the guide of the device conveniently limits the degrees of freedom of movement of the surgical instrument relative to the joint part fixed on the spherical support surface, which makes handling much easier for the operator. Intraoperative alignment times are minimized by shifting them to the preoperative phase. For complex surgeries, the option is provided to use a manipulator or robot as a surgical aid to assist in the surgery, so that new fields of application are opened up for the device provided herein as compared to the treatment aids known in the prior art.

In this connection, it is particularly advantageous if the support surface is releasably connected to the device, in particular by a quick-release fastening means, so as to allow positioning to be performed independently of the fixation procedure. To this end, the joint part is initially fixed on the support surface, which in turn is then positioned on the device by means of the quick-release fastening means. For this purpose, the quick-release fastening means has only a single fixing element, in particular a quick-acting clamp, and can be fixed on the device by a form-locking connection, in particular a snap-fit connection.

In a particularly advantageous embodiment of the device according to the present invention, the guide has a slotted opening for the surgical instrument, in particular an oscillating or piezoelectric saw, or for surgical aids, in particular measurement means. This allows easy adjustment of the desired working plane, enabling its angular position relative to the joint parts to be adjusted with reproducible accuracy.

It is particularly practical if the guide allows the surgical instrument or the surgical aid to be moved with only one degree of freedom, so as to thereby provide, for example, an axial sliding guide to provide valuable assistance to the operator.

The receiving means could have generally known connecting elements of any type, for example also force-fitting connecting elements. However, it is particularly useful if the receiving means has a projection which engages in a groove. This does not only allow a fixed angular position of the guide with respect to the joint parts to be adjusted with little effort by the orientation of the groove, but also enables the distance to be variably adjusted by moving the projection toward the joint within the groove.

It is particularly practical if the receiving means has a plurality of grooves which can be selected as needed. This allows a particularly flexible use of the device for adaptation to different joint parts or for individual adaptation to the patient to be treated.

In another particularly advantageous embodiment of the device of the present invention, the device has a plurality of receiving means, at least one support surface of each said receiving means being associated with the distal joint part and the proximal joint part so as to allow both joint parts to be treated without changing the support. Here, the receiving means are identical in terms of their geometry and orientation relative to the associated support surface, thus allowing alternative fixation options within the same guide.

In order for the guide to also be orientable as desired relative to the respective joint part, it is advantageous if the projection is pivotably disposed on the guide. To this end, the guide may be configured as a substantially cylindrical component having a concentric mounting arrangement which is located in the end region and carries the groove. The axis of rotation, which can be provided in this manner, advantageously extends parallel to the axis of rotation of the joint.

In a particularly useful embodiment of the device of the present invention, the guide can be fixed in position at different distances with respect to the joint, so as to be able, for example, to suitably adjust the depth of treatment for a particular surgical instrument.

A particularly reliable fixation of the two joint parts on the respectively associated support surface is achieved because the holding element has a fixing means which can be locked in different positions. This allows the operator to individually select the point of contact of the fixing means with the respective joint part, so as to ensure optimum treatment.

For this purpose, in a particularly simple embodiment, the fixing means has a threaded portion which can be screwed into a threaded socket of the holding element, so that opposing fixing means become fixed between the respective joint part by screwing them into the holding element. Such fixing elements, which may also be in the form of bone screws, may therefore be screwed into a plurality of threaded sockets in the holding element as needed and in different numbers.

The fixing means has a conical portion which is associated with the joint part and which, as an end portion, is located adjacent the threaded portion on the side of the joint part, and which thus allows the joint part to be reliably fixed in place. The conical portion may be attached rotatably relative to the fixing means such that it may rotate about the screw rotational axis thereof, which prevents rotary motion from being transmitted to the conical portion. Of course, the conical portion may also be a separate component having a threaded socket to receive a threaded stud supported on the holding element, so that no threaded sockets are needed on the holding element.

Another particularly practical embodiment of the present invention is one in which the support surface, the holding elements, and the receiving means for the guide together form a unit which is mounted on a base plate of the device in such a manner that it can pivot about an axis parallel to the axis of the joint. On the one hand, this provides a secure support for the device including the joint fixed in place therein; on the other hand, the pivotable attachment allows for optimal alignment of the joint for subsequent treatment.

Moreover, it is particularly useful if two support regions of the support surface are inclined relative to each other so as to allow the two joint parts to be supported in a position between the flexed position and the extended position of the joint, so that each joint part is supported in an optimum manner. The support regions may be fixed in different positions relative to each other, in particular at different angles of inclination and, in another advantageous variant, they may be connected by a spherical region which supports the joint in a predetermined manner.

It is also particularly practical for the device to have markings, in particular a scale for adjustment of the relative position of the guide with respect to the joint, so that reproducible results can be obtained.

It is also useful if, during the surgical procedure, at least some of the relatively movable elements of the guide or of the fixing means can be restrained, in particular fixed in position, by a stop to prevent accidental displacement, and to thereby prevent accidental deviation from a predetermined area of treatment.

### BRIEF DESCRIPTION OF THE DRAWINGS

The present invention may be embodied in different forms. To further illustrate its basic principle, one embodiment thereof is shown in the drawing and will be described hereinbelow. In the drawing,

FIG. 1 is a perspective view of a device according to the present invention, including a guide;

FIG. 2 is an enlarged perspective detail view of the device shown in FIG. 1;

FIG. 3 is a perspective view of two holding elements of the device, which are each provided with a plurality of fixing means;

FIG. 4 is a perspective view of another device according to the present invention.

### DETAILED DESCRIPTION

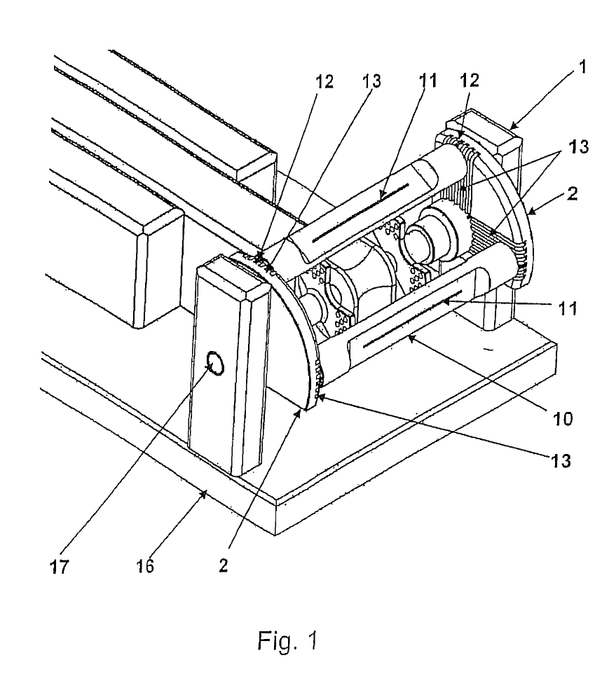

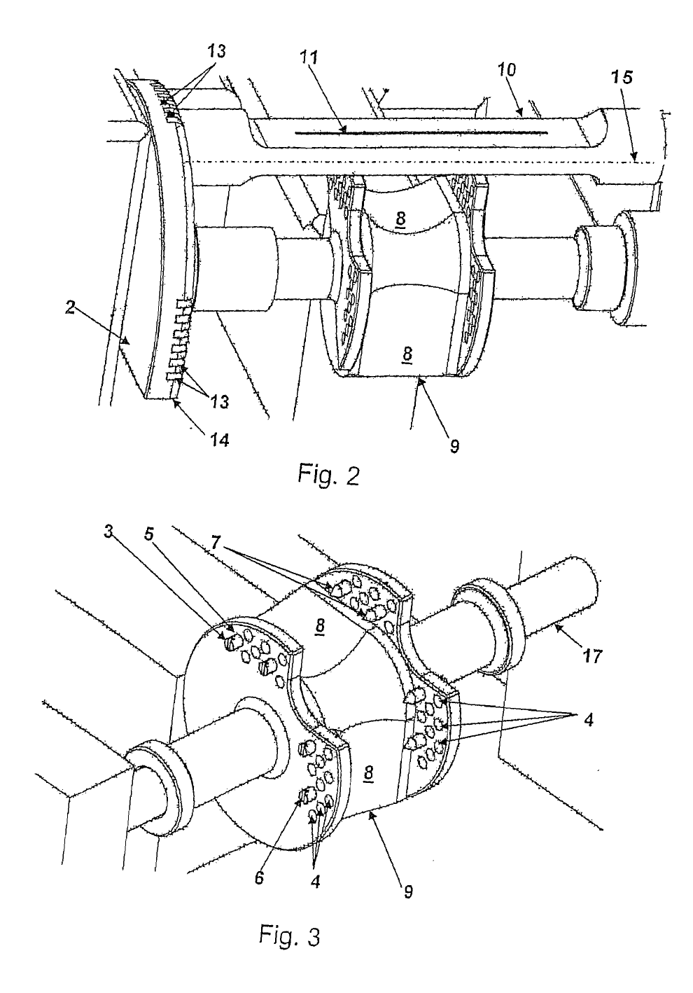

A device 1 according to the present invention, which is intended to be used for a joint, is illustrated in greater detail in the perspective views of FIGS. 1 through 3. In order for device 1 to fix in place a distal part and a proximal part of a joint, it has two holding elements 2 which are disposed opposite one another and between which the respective part of the joint can be fixed in place by means of a releasable fixing means 3. For this purpose, fixing means 3 has a plurality of bone screws 6 which can each be screwed with a threaded portion 5 into a corresponding threaded socket 4 of holding element 2 and which each have a conical portion 7 which is associated with the joint part. In order to support the two joint parts in a flexed position, support surface 9 has two concave support regions 8 which are inclined relative to each other. Device 1 further has a guide 10 having a slotted opening 11 for a surgical instrument (not shown), which conveniently limits the degree of freedom of movement of the surgical instrument so as to reduce stress on the operator. In order to allow guide 10 to be positioned as needed, it can be inserted by two projections 12 into one of a plurality of parallel grooves 13 of a receiving means 14 of device 1, and can be fixed in position between holding means 2 by axially tightening the same. This also adjusts the angular position of the guide 10, which is pivotable about its longitudinal center axis 15 relative to projections 12. In order to simplify the handling of device 1, and to facilitate the surgical procedure, support surface 9, holding elements 2, and receiving means 14 for the guide 10 together form a unit which is mounted on a base plate 16 of device 1 in such a manner that it can pivot about an axis 17.

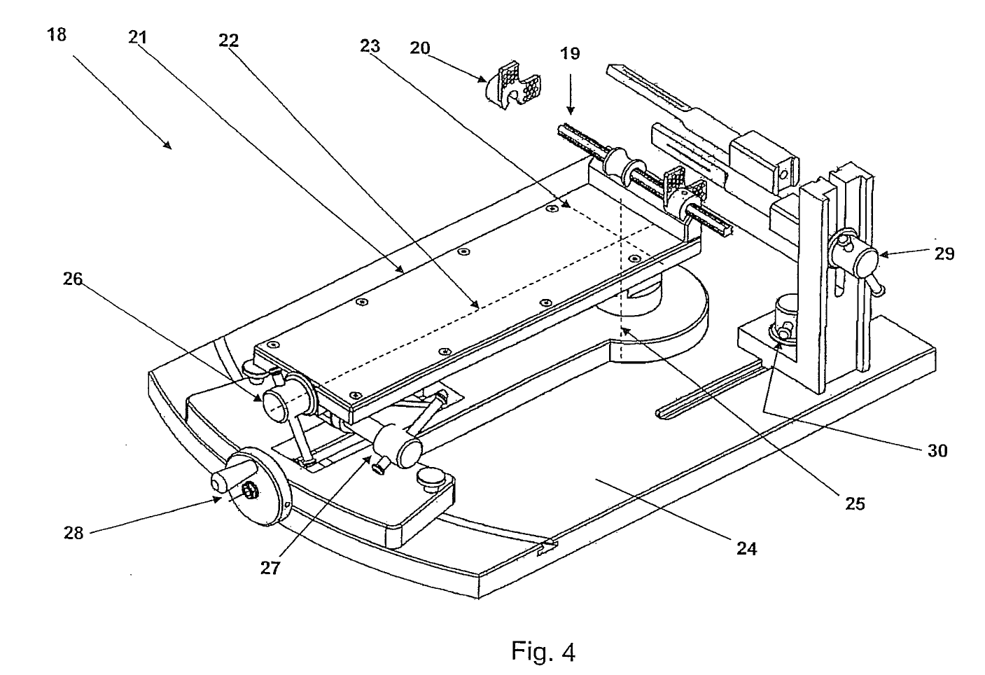

Another device 18 according to the present invention is shown in a perspective view in FIG. 4. In this embodiment, in order for device 18 to fix in place a distal part and a proximal part of a joint (not shown), it has two holding elements 20 which are disposed opposite one another and are relatively movable on a common shaft 19 in the form of a splined shaft, and between which the respective part of the joint can be fixed in place by means of a releasable fixing means. For this purpose, the fixing means has a plurality of bone screws 6 which can each be screwed into a corresponding threaded socket 4 of the respective holding element 20. In order to support the two joint parts in a flexed position, support surface 9 has two concave support regions 8 which are inclined relative to each other and connected to a common forearm support 21. Forearm support 21 is pivotable about two axes 22, 23 which extend perpendicularly to each other in the plane of forearm support 21 and about an axis 25 which is perpendicular to a base plate 24 of device 18, said forearm support further being fixable in the desired position by means of an adjustment device 26, 27, 28, respectively. Device 18 further has a guide 10 having a slotted opening 11 for a surgical instrument (not shown), which can similarly be fixed in the desired vertical position by an adjustment device 29, and in different positions on base plate 24 by a further adjustment device 30.

What is claimed is:

1. A device for temporary fixation of parts of a human joint having a distal joint part and a proximate joint part, the device comprising:
two holding elements disposed opposite one another;
a releasable fixing device configured to fix in place a respective joint part of the human joint between the two holding elements;
a support surface configured to support the distal joint part and the proximal joint part;
a guide configured to position and to limit a relative movement of at least one of a surgical instrument and a surgical aid; and
a receiving means for receiving the guide.

2. The device as recited in claim 1, further comprising a quick-release fastening means for releasably connecting the support surface with respect to the holding elements.

3. The device as recited in claim 2, wherein the quick-release fastening means includes a fixing screw.

4. The device as recited in claim 3, wherein the quick-release fastening means includes a snap-fit connection.

5. The device as recited in claim 1, wherein a position of at least one of the holding elements is adjustable so as to adjust a spacing between the two holding elements.

6. The device as recited in claim 1, wherein at least one of the holding elements is movable along an axis and can be fixed in position by a force-fit connection.

7. The device as recited in claim 6, wherein the holding elements are mounted such that they can be moved together along the axis.

8. The device as recited in claim 1, wherein the guide has a slotted opening receiving the surgical instrument or surgical aid.

9. The device as recited in claim 8, wherein the surgical instrument includes one of an oscillating and a piezoelectric saw and wherein the surgical aid includes a measurement device.

10. The device as recited in claim 1, wherein the guide limits the surgical instrument or the surgical aid to one degree of freedom.

11. The device as recited in claim 1, wherein the receiving means includes a projection engaging in a groove.

12. The device as recited in claim 11, wherein the receiving means includes a plurality of grooves, and the projection can be alternatively inserted in one of the plurality of grooves.

13. The device as recited in claim 11, wherein the projection is pivotably disposed on the guide.

14. The device as recited in claim 1, further comprising a plurality of receiving means, and wherein the support surface includes two support regions, each being associated with one of the distal joint part and the proximal joint part.

15. The device as recited in claim 1, wherein the guide can be fixed in position at different distances with respect to the joint.

16. The device as recited in claim 1, further comprising an eccentric adjustment means configured to fix the guide in different positions relative to a longitudinal center axis of the guide.

17. The device as recited in claim 1, wherein the guide includes two guide regions, each associated with one of the distal and proximate joint parts and adjustable independently of each other.

18. The device as recited in claim 1, wherein the fixing devices can be fixed in different positions on the holding element.

19. The device as recited in claim 1, wherein the fixing device includes a threaded portion screwable into a threaded socket of the holding element.

20. The device as recited in claim 1, wherein the fixing device has a conical portion associated with the respective joint part.

21. The device as recited in claim 1, further comprising a base plate, and wherein the support surface, the holding elements, and the receiving means form a unit that is mounted on the base plate so that the unit is pivotable about an axis parallel to an axis of the joint.

22. The device as recited in claim 1, wherein the support surface includes two support regions inclined relative to each other so as to allow the two joint parts to be supported in a position between a flexed position and an extended position of the joint.

23. The device as recited in claim 22, wherein the support surface includes a spherical region connecting the two support regions.

24. The device as recited in claim 1, further comprising has markings forming a scale for adjustment of a relative position of the guide relative to the joint.

25. The as device as recited in claim 1, further comprising a stop configured to restrain a relative movement of at least one of the guide and the fixing device so as to prevent an accidental displacement.

26. The device as recited in claim 1, wherein the support surface is fixable in a desired position and independently movable about three axes.

27. The device as recited in claim 1, further comprising a forearm support connected to the support surface.

28. The device as recited in claim 27, wherein the forearm support is pivotable about two axes extending perpendicularly to each other in a plane of the forearm support and about a third axis perpendicular to a base plate of the device.

29. The device as recited in claim 1, further comprising an instrument for detecting a relative offset of a plurality of compartments of a joint part.

30. The device as recited in claim 1, wherein the human joint is a joint of one of a hand and a finger.
